(19) United States
(12) Patent Application Publication
(10) Pub. No.: US 2005/0021154 A1
(43) Pub. Date: Jan. 27, 2005

(54) Drive device for a finger prosthesis

(76) Inventor: Stellan Brimalm, Skanningegatan (SE)

Correspondence Address: VENABLE, BAETJER, HOWARD AND CIVILETTI, LLP, P.O. BOX 34385, WASHINGTON, DC, 20043-9998, US

(21) Appl. No.: 10/488,008

(22) PCT Filed: Aug. 26, 2002
(86) PCT No.: PCT/SE02/01511

(30) Foreign Application Priority Data
Aug. 27, 2001 (SE) 0102833-1

(51) Int. Cl. A61F 2/48

(52) U.S. Cl. 623/64; 623/24

(57) ABSTRACT

Drive device (1) for a finger prosthesis (2) of substantially natural size designed to bend the finger prosthesis (2) about a shaft (A) relative to a fixing (3) e.g. in a human or artificial metacarpus (4). The drive device (1) comprises a motor (6) which can be connected to an energy source e.g. a battery, and a transmission (5, 7, 8) intended to transform a force from the motor (6) to the finger prosthesis (2) to perform the movement. The motor (6) and transmission (5, 7, 8) are placed in the finger prosthesis (2) so that the bending shaft (A) is contained in the finger prosthesis (2) or in its extension at the fixing (3).

### TECHNICAL FIELD

The present invention concerns a device for operating the movement of a finger prosthesis. The finger prosthesis has a natural size and is intended to be attached to a metacarpus prosthesis or a human hand. The finger prosthesis and metacarpus prosthesis are intended to replace a corresponding lost human body part.

### BACKGROUND OF THE INVENTION AND PROBLEM

When a hand or metacarpus and fingers are lost or amputated, they can be replaced by a corresponding prosthesis. This means a loss of a functioning body part with realistic appearance and a movement pattern which a prosthesis must simulate.

The prosthesis to replace the lost body part should be simple to repair and also have a cost-effective construction so that e.g. the supply of spare parts can be improved.

Some known finger prostheses have a drive device with means for transmitting energy from a motor arranged at the wrist to the finger prosthesis, which means e.g. contain wires to achieve a bending of the fingers. On failure of a finger prosthesis the fault must first be located in the wrist, finger prosthesis or transfer means, where upon the part concerned must be repaired or replaced. Arranging the motors and drive elements in an artificial metacarpus reduces the possibility of arranging other organs therein.

The finger prosthesis must also have as natural a bending movement as possible and be gentle towards a prosthesis cover consisting of an elastic material and arranged finally over the finger prosthesis to simulate human skin.

Specification U.S. Pat. No. 5,888,246 discloses a device for bending a finger prosthesis. The device has a motor and drive/gear elements designed to bend the entire finger prosthesis. The text also describes a worm gear to transform the motor rotation into the finger prosthesis movement.

### OBJECT OF THE INVENTION AND SOLUTION OF THE PROBLEM

The object of the invention is to solve one or more of the above problems.

This is achieved with a device according to claim 1.

By integrating a drive device, comprising a motor and transmission intended to transform the motor movement into finger movement, in a finger prosthesis designed to bend the finger prosthesis about a shaft in relation to a fixing, e.g. in a metacarpus prosthesis or human hand, in a movement direction from an extended position towards a palm and out again to the extended position, a finger prosthesis with substantially natural size is obtained which is easy to replace and allows a simplified repair of the finger prostheses. The motor can be connected to an energy source e.g. a battery.

Furthermore by placing the motor and transmission in the finger prosthesis, importantly space is created at other points e.g. in a metacarpus prosthesis, which means that the metacarpus prosthesis e.g. can be made smaller and/or contain other equipment e.g. control devices or batteries.

Furthermore by dividing the finger prosthesis into two parts pivotable against each other and coupling the movement of these parts together, a simulation of a human finger in appearance and movement is achieved.

### DESCRIPTION OF THE DRAWINGS

The invention will be described further in an embodiment example with reference to the figures as follows.

FIG. 1 shows a diagrammatic cross-section sketch from the side of a drive device according to the invention for a finger prosthesis.

FIG. 2 shows a diagrammatic sketch of an angled gear according to the invention for a finger prosthesis.

FIG. 3 shows a diagrammatic cross-section sketch from the side of a drive device according to the invention with a pantograph rod for a finger prosthesis in extended position.

FIG. 4 shows a diagrammatic cross-section sketch from the side of a drive device according to the invention with a pantograph rod for a finger prosthesis in the bent position.

FIG. 5 shows a diagrammatic sketch of a drive device according to the invention in a finger prosthesis attached to a hand prosthesis.

FIG. 6 shows a hand prosthesis according to FIG. 5b with the finger prostheses bent towards the palm.

FIG. 7 shows a diagrammatic sketch, in a view towards a palm, of a drive device according to the invention in a finger prosthesis fastened to a hand prosthesis.

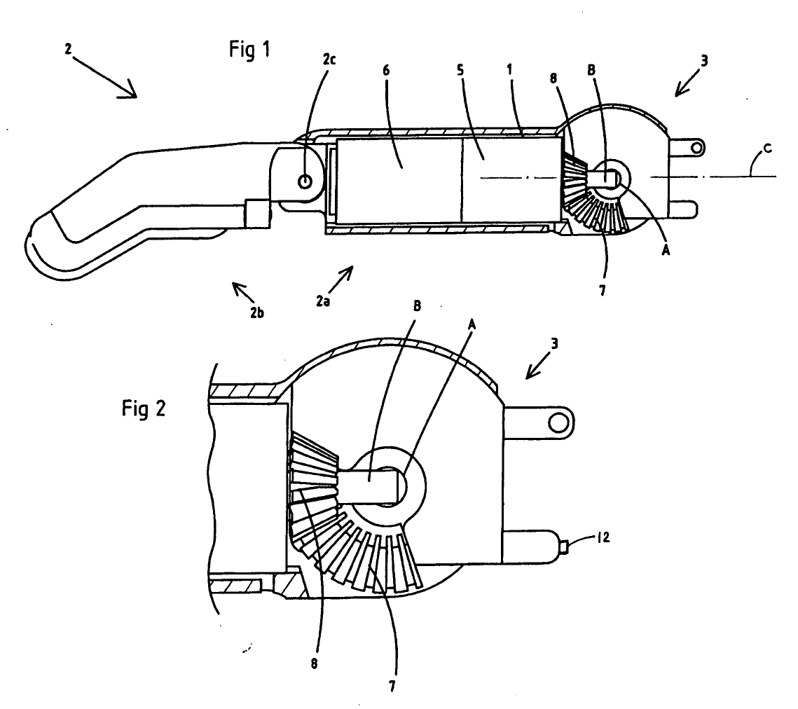

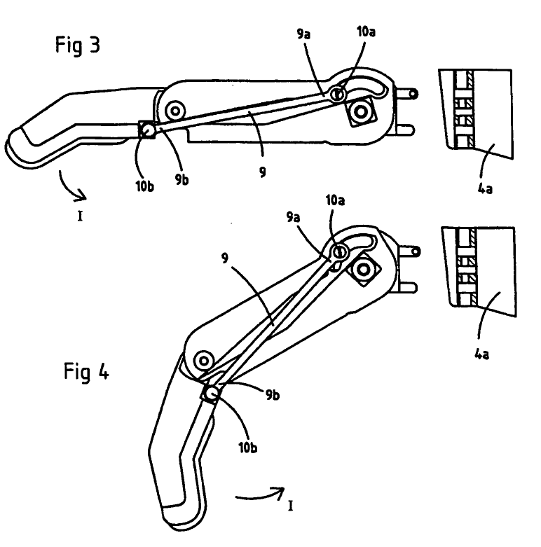

### DESCRIPTION OF EMBODIMENT EXAMPLE

A drive device for bending a finger prosthesis is referred to generally as 1, where the finger prosthesis is referred to as 2. A fixing 3 is designed to attach the finger prosthesis to a human or artificial metacarpus 4. Substantially the entire finger prosthesis is designed to bend relative to the fixing 3 to simulate a human finger movement. Furthermore the finger prosthesis has a first part 2a and a second part 2b which are connected at a finger joint 2c so that the parts can move in relation to each other to further simulate a human finger movement. The finger prosthesis 2 is intended to bend about a shaft A placed at the fixing 3. A transmission 5 contains a gearbox 5b and an angled gear 5a. The angled gear 5a is arranged at shaft A and preferably has two bevel gear wheels 7, 8, of which the first gear wheel 7 has teeth at least partly about shaft A, and the other gear wheel 8 has teeth around an outgoing shaft B from gearbox 5b. The first gear wheel 7 in the preferred embodiment is designed as a bevel gear wheel describing a part smaller than a complete rotation. As the finger is to be turned/bent around shaft A through 70 to 100 degrees, the gear wheel 7 describes around 120 degrees. Shafts A, B are arranged substantially in the same plane but in an alternative embodiment of the angled gear 5a, the second gear wheel 8 can be arranged slightly offset along the edge of the first gear wheel 7, e.g. by means of a hypold gear, where the planes of the shafts do not lie in the same plane but close to each other. At gearbox 5b is also a motor 6. The motor 6 and gearbox 5b are mounted together and housed in the finger prosthesis 2 between shaft A and the finger joint 2c. The gearbox 5b is advantageously of the trochoid type. The motor 6 is preferably an electric motor and can therefore be connected by means of a power supply lead to a battery. The central shaft of the motor coincides substantially with a central axis C for the finger which extends in the linear centre and along the extent of the finger. The gearbox 5b and the two bevel gear wheels 7, 8 are designed to transform the energy of motor 6 into finger movement and hence force in the finger prosthesis. As the finger prosthesis 2 contains the motor 6 and the two bevel gear wheels 7, 8, the finger prosthesis 2 can quickly be detached from e.g. the metacarpus prosthesis 4 by opening the fixing and releasing an electrical contact 12 for the power supply, thus simplifying e.g. exchange of finger 2 for repair.

The finger prosthesis 2 is substantially of natural size and is designed to give a natural movement pattern. Thus the finger prosthesis 2, referred to below in the description as the finger 2, is formed from a substantially circular tube, preferably of a light and strong material e.g. aluminium or reinforced plastic. Furthermore both shaft A and finger joint 2c are arranged substantially in the centre of finger 2 at a central axis C or in an alternative embodiment at least within ±15 mm from the centre axis C to give a natural movement pattern. On use, finger 2 usually has a skin-simulating prosthesis cover (not shown). By arranging finger joint 2c and/or shaft A substantially in the middle of the hollow tube or its extension, advantageously on finger movement the prosthesis cover stretches on the outside of the joint or fastening 3 i.e. at the knuckle and the cover is compressed on the inside i.e. on the side of finger 2 which on bending is on the inside on bending direction I. This reduces the maximum load on the prosthesis cover.

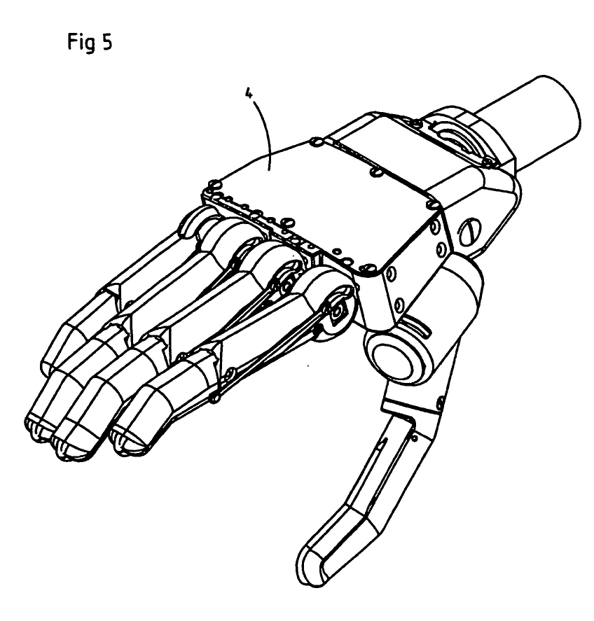

A pantograph rod 9 is arranged preferably on each side of the finger prosthesis, see FIGS. 3 and 4. The pantograph rod 9 is attached pivotably at the first end 9a to the fixing 3 of the prosthesis finger at a point 10a which does not move with the prosthesis on bending. The other end 9b of the pantograph rod 9 is pivotably connected to the other part 2b of the finger prosthesis at a point on this part 2b located before the finger joint on the inside viewed in the bend direction I for the finger prosthesis towards an artificial or human palm. On extension of the prosthesis finger 2 therefore the pantograph rod 9 has a compressive effect and presses the other finger prosthesis part 2b towards an extended position, see FIG. 5b. The bend movement between the first and second prosthesis parts 2a and 2b is thus controlled by the bending movement between the first part 2a of the finger prosthesis relative to fixing 3, see FIGS. 3 to 6. Operating both these bending movements at the same time with a common motor 6, gearbox 5b and angle gear gives a simple and robust prosthesis designed to simulate a human finger and its movement. In the preferred embodiment the finger prosthesis 2 has two pantograph rods which are arranged on either side of the finger prosthesis 2.

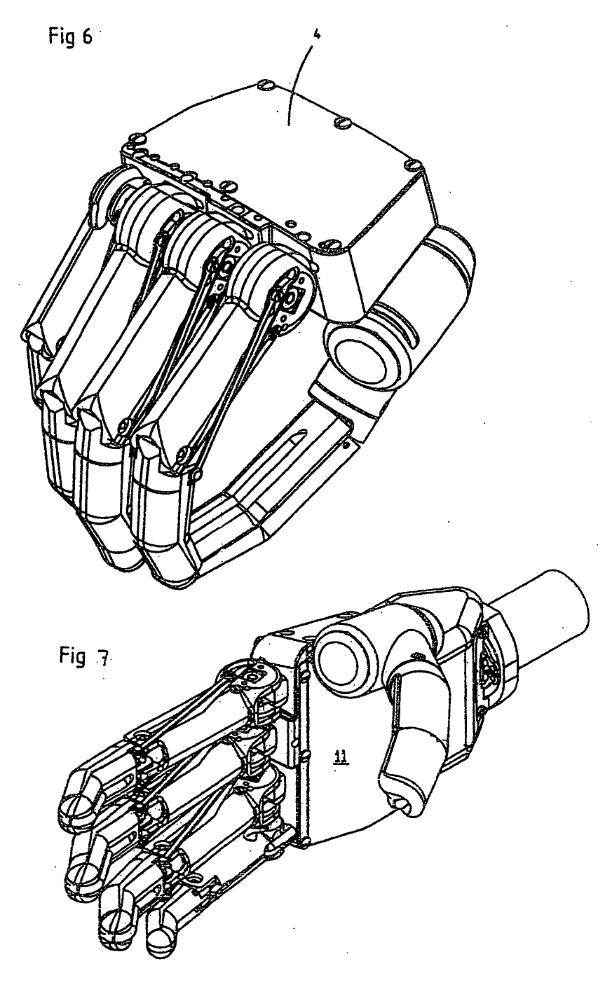

The finger prostheses as above can be arranged next to each other e.g. to replace the lost human index, middle and ring fingers as shown in FIGS. 5b and 6.

The invention should not be regarded as limited to the examples described above but can vary within the scope of the claims.

List of References

1 drive device

2 finger prosthesis

2a first part of finger prosthesis

2b second part of finger prosthesis

3 fixing

4 metacarpus

4a part of metacarpus

5 gearbox

6 motor

7 first gear wheel

8 second gear wheel

9 pantograph rod

9a first part of pantograph rod

9b second part of pantograph rod

10a fixing point at fixing

10b fixing point at other prosthesis parts

11 palm

12 contact

I Bend angle

A Bend shaft

B Gearbox shaft

C Centre axis

1. A drive device with a finger prosthesis of substantially natural size designed to bend a finger prosthesis about a shaft in relation to a fixing at a human or artificial metacarpus at a bending angle from an extended position towards a palm and out again to the extended position, where the drive device comprises a motor which can be connected to an energy source e.g. a battery, and a transmission designed to transform power from the motor to the finger prosthesis to perform the bending, wherein the motor and the transmission are placed in the finger prosthesis so that the bending shaft is contained in the finger prosthesis or its extension at the fastening.

2. The drive device according to claim 1, wherein the bending shaft is arranged within 15 mm from a center axis which extends along the center of the finger prosthesis.

3. The drive device according to claim 1, wherein the transmission a first drive wheel connected with the motor and a second drive wheel connected with the fastening, which drive wheel is designed under co-operation with others to transfer the force from the motor to the finger prosthesis to perform the movement, where the drive wheels are arranged on shafts which lie substantially in the same plane.

4. The drive device according to claim 1, wherein the finger prosthesis comprises a first and a second prosthesis part which are connected together by articulation, that the transmission furthermore comprises pivoting means to pivot the second finger prosthesis part relative to the first finger prosthesis part in towards the palm.

5. The drive device according to claim 4, wherein the pivoting means comprise a pantograph rod which has a first articulated fastening at the finger prosthesis fixing and extends past the first part of the finger prosthesis and has a second articulated fixing in the second part of the finger prosthesis at a point on this part which, on tension in the pantograph rod, causes bending in towards the palm.

6. The drive device according to claim 1, wherein the motor is an electric motor and that the energy source is arranged separately from the finger prosthesis e.g. in the metacarpus, that at the fixing is a contact for dividing the power supply arranged between the motor and the energy source.
